(19) United States
(12) Patent Application Publication
(10) Pub. No.: US 2022/0051775 A1
(43) Pub. Date: Feb. 17, 2022

(54) METHOD FOR PROVIDING CUSTOMIZED MEDICATION FOR WEIGHT LOSS

(71) Applicant: Joe Duarte Group, LLC, Belmont, MI (US)

(72) Inventor: Nishant A. Rao, La Crescenta, CA (US)

(21) Appl. No.: 17/444,928

(22) Filed: Aug. 12, 2021

Related U.S. Application Data
(60) Provisional application No. 63/064,680, filed on Aug. 12, 2020.

(51) Int. Cl. G16H 20/10; G16H 50/30; G16H 10/60; A61K 45/06

(52) U.S. Cl. CPC G16H 20/10; A61K 45/06; G16H 10/60; G16H 50/30

(57) ABSTRACT

A method for determining a customized medication for weight-loss including a variable composition. The variable composition includes one or more additive ingredients selected from a plurality of additive ingredients. The method includes the steps of displaying a plurality of questions to a user, receiving user responses from the user corresponding to each question in the plurality of questions, analyzing the user responses, disqualifying any of the additive ingredients in the plurality of additive ingredients based on the user responses, and creating a customized medication for the user once a user response has been received for each of the questions in the plurality of questions. The customized medication includes any of the additive ingredients in the variable composition which have not been disqualified.

### CROSS REFERENCE TO RELATED APPLICATION

This application claims the benefit of U.S. Provisional Application 63/064,680, which was filed on Aug. 12, 2020, the disclosure of which is hereby incorporated by reference.

### BACKGROUND OF THE INVENTION

### 1. Field of the Invention

The present invention generally relates to a method and system for selecting medication for weight loss. More particularly, the present invention pertains to a method of determining an optimal weight-loss prescription/medication for a patient based upon a patient's information.

### 2. Description of the Prior Art

In the United States, around sixty percent of men and fifty-two percent of women, with the age of 20 years or older, are overweight or obese. In addition, a large percentage of children in the United States are overweight or obese. Two approved weight loss medications, Contrave® and Qsymia®, contain between them the primary prescription compounds that are generally prescribed for weight loss. However, these two medications both have significant drug/drug interactions and contraindications based on prior medical history. Patients seeking these interventions are often denied for safety reasons with few other options. The method and system described hereinbelow will facilitate in identifying which of the chemicals/compounds would be safe for a patient, and accordingly determine an optimal weight loss prescription for the patient.

### SUMMARY OF THE INVENTION

According to an aspect of the disclosure a method for determining a customized medication for weight-loss is disclosed. The customized medication includes a base composition and a variable composition including one or more additive ingredients, wherein the one or more additive ingredients are selected from a plurality of additive ingredients. The method includes displaying, by a display, a plurality of questions to a user, and receiving, by an input device, user responses of the user corresponding to the displayed questions. The method further includes analyzing, by the processor, the user responses, and disqualifying, by the processor, any of the additive ingredients in the plurality of additive ingredients based on the user responses.

Optionally, the base composition includes one or more main ingredients being selected from the group consisting of 5-HTP, caffeine, DHEA, cyanocobalamin, methionine, inositol, chromium, and carnitine.

Optionally, the base composition includes each of the main ingredients.

Optionally, the plurality of additive ingredients includes naltrexone, yohimbine, bupropion, topiramate, phentermine, and metformin.

Optionally, each of the questions in the plurality of questions relates to whether the user should be disqualified from using one or more of the additive ingredients of the variable composition.

Optionally, the method for determining a customized medication can include the step of creating, by the processor, of a customized medication for the user once a user response has been received for each of the questions in the plurality of questions, the customized medication including the base composition and any of the additive ingredients in the variable composition which have not been disqualified by the processor.

According to another embodiment hereof, there is disclosed a method for determining a customized medication for weight-loss. The customized medication includes a variable composition including one or more additive ingredients, wherein the one or more additive ingredients are selected from a plurality of additive ingredients. The method includes displaying, by a display, a plurality of questions to a user, and receiving, by an input device, user responses of the user corresponding to the displayed questions. The method further includes analyzing, by the processor, the user responses, and disqualifying, by the processor, any of the additive ingredients in the plurality of additive ingredients based on the user responses.

Optionally, the plurality of additive ingredients includes naltrexone, yohimbine, bupropion, topiramate, phentermine, and metformin.

Optionally, each of the questions in the plurality of questions relates to whether the user should be disqualified from using one or more of the additive ingredients of the variable composition.

Optionally, the method for determining a customized medication can include the step of creating, by the processor, of a customized medication for the user once a user response has been received for each of the questions in the plurality of questions, the customized medication including any of the additive ingredients in the variable composition which have not been disqualified by the processor.

For a more complete understanding of the present invention, reference is made to the following detailed description and accompanying drawings. In the drawings, like reference characters refer to like parts throughout the views in which:

### BRIEF DESCRIPTION OF THE DRAWINGS

FIG. 1 illustrates a system that facilitates in determining a weight-loss prescription based on a patient's information, in accordance with an embodiment of the disclosure; and

FIG. 2 illustrates a method for determining a weight-loss prescription for a patient, in accordance with an embodiment of the disclosure.

### DETAILED DESCRIPTION OF A PREFERRED EMBODIMENT

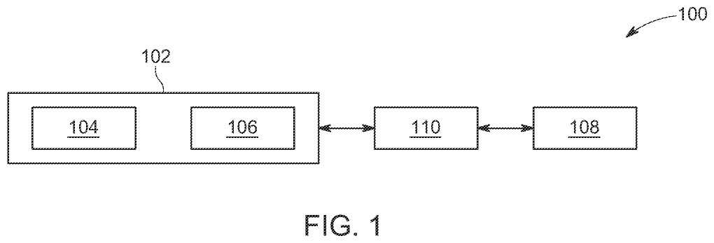

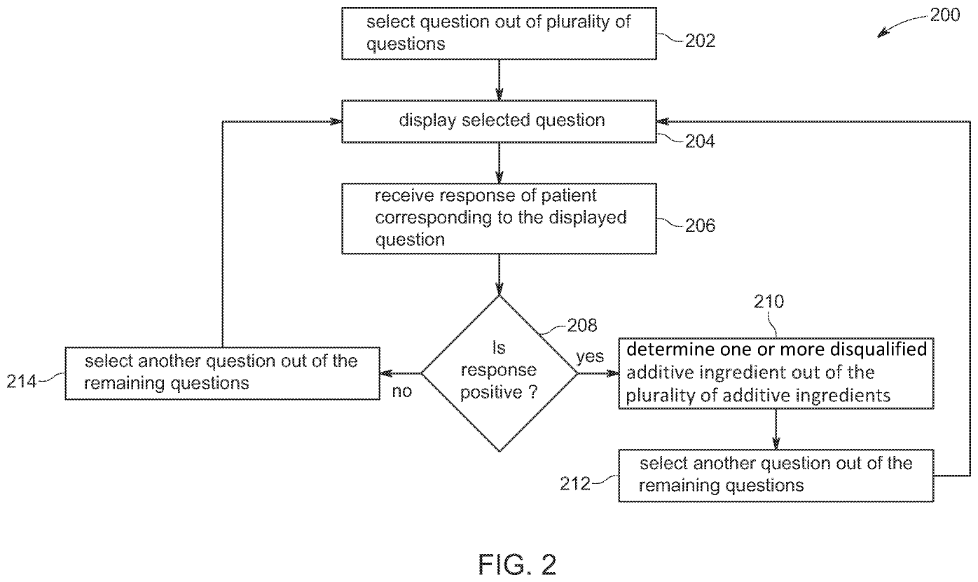

Referring to FIGS. 1 and 2, a system and a method to determine a customized weight-loss medication/prescription (hereinafter referred to as medication) is shown. The medication is a combination of a fixed base composition having a plurality of main ingredients, and a variable composition having a plurality of additive ingredients. It may be appreciated that the system and the method facilitate in determining the variable composition of the customized medication, while keeping the base composition unchanged. In an embodiment, the fixed base composition includes a 5-HTP, a caffeine, a DHEA, a cyanocobalamin, a methionine, an inositol, a chromium and a carnitine. The plurality of main ingredients, if consumed in a predefined quantity, facilitate in reducing and maintaining the weight of the individual. As an example, the predefined quantity of the main ingredients may include 25 milligrams (mg) of 5-HTP, 25 mg of caffeine, 10mg of DHEA, 500 micrograms (mcg) of cyanocobalamin 500, 25 mg of methionine, 25 mg of inositol, 25 mcg of chromium, and 100 mg of carnitine.

Further, the variable composition includes the plurality of additive ingredients, for example, naltrexone, yohimbine, bupropion, topiramate, phentermine, and metformin It may be appreciated that the plurality of additive ingredients is adapted to work upstream and centrally in the brain to help in reducing food cravings and regulating an appetite. However, one or more additive ingredients of the plurality of additive ingredients may be not suited to the patient. To determine the suitability of each of the additive ingredients of the variable composition, a plurality of questions are presented to a patient. Each question in the plurality of questions relates to, or is intended to determine, whether the patient is to be disqualified from using one or more of the additive ingredients of the variable composition.

In an embodiment, the medication does not necessarily include the fixed base composition. According to this embodiment, the medication is determined by evaluating whether or not any of the additive ingredients in the variable composition should be prescribed or disqualified as discussed hereinbelow, and none of the main ingredients are necessarily required or prescribed.

In an embodiment, the plurality of questions includes a question to confirm if the patient is taking any narcotic or opioid pain medications, or has severe allergies to opioid drugs to check a disqualification of usage of naltrexone by the patient. Further, the plurality of questions includes a question to confirm if the patient is suffering from any kidney or liver disease to check a disqualification of usage of naltrexone and topiramate by the patient. Also, the plurality of questions includes a question to confirm if the patient is taking any immunosuppressant drugs to check a disqualification of usage of naltrexone by the patient.

Moreover, the plurality of questions includes a question to confirm if the patient is currently taking any medications for high blood pressure to check a disqualification of usage of yohimbine by the patient. The plurality of questions also includes a question to confirm if the patient has uncontrolled hypertension to check a disqualification of usage of naltrexone by the patient. The plurality of questions also includes a question to confirm if the patient is currently taking any stimulants to check a disqualification of usage of yohimbine by the patient.

Furthermore, the plurality of questions includes a question to confirm if the patient is taking any antidepressant medications to check a disqualification of usage of topiramate and bupropion by the patient. The plurality of questions also includes a question to determine if the patient has a history of seizures or a seizure disorder to check a disqualification of usage of bupropion by the patient. The plurality of questions also includes a question to confirm if the patient has any history of anorexia or bulimia to check a disqualification of usage of bupropion by the patient.

Further, the plurality of questions includes a question to confirm if the patient is taking any medications for migraines to check a disqualification of usage of topiramate by the patient. The plurality of questions also includes a question to determine if the patient has any bleeding disorders to check a disqualification of usage of topiramate by the patient. The response of each of the plurality of questions may be a positive response or a negative response. As an example, the individual can answer any of the questions by responding “yes” accepted as a positive response or by responding “no” accepted as a negative response. Based upon the responses of the individual, a right combination of the additive ingredients is determined, and the customized medication is prescribed.

Again, referring to FIG. 1, the system 100 includes a user interface 102 adapted to interact with the patient and having a display 104 and an input device 106, such as a keyboard, a memory 108 adapted to store the plurality of questions, and a processor 110 in communication with the user interface 102 and the memory 108, and adapted to display the questions to the patient via the display 104. The processor 110 is further adapted to receive the answers/inputs corresponding to the questions displayed to the patient, and determine the disqualification of one or more additive ingredients based on the received user inputs corresponding to the questions. Consequently, the processor 110 determines which additive ingredients can be consumed by the patient, thus determining the contents of the variable composition. Accordingly, the processor 110 determines the customized medication to be used by the patient for weight-loss.

Now referring to FIG. 2, the method 200 for determining the customized medication for the patient is shown. The method 200 includes a step 202. At step 202, the processor 110 may select a question from the plurality of questions. In some embodiments, the processor 110 may select the question from the plurality of the questions based on one or more personal details of the patient. The plurality of personal details may include, but are not limited to, name, gender, date of birth (DOB), height, and/or weight of the patient. In such a case, the processor may collect one or more personal details of the patient before displaying the question to the patient. For collecting the personal details, the processor 110 may show, via the display 104, a template to the patient and prompts the patient to enter the one or more personal details inside the template. The patient may then enter the personal details via the input device 106. The personal details may be stored within the memory 108 by the processor 110.

After selecting the question from the plurality of questions, the method 200 moves to a step 204, in which the processor 110 may show the selected question to the patient, via the display 104. For example, the display 104 may display a question corresponding to determining if the patient is taking any narcotic or opioid pain medications, or have severe allergies to opioid drugs. The processor 110 may then prompt the patient to enter a response to the question. Subsequently, at step 206, the patient enters the response of the question via the input device 106. Upon receiving the response of the question, the processor 110 may store the response in the memory 108, and the method 200 moves to a step 208. At step 208, the processor 110 analyzes the response of the patient. The method 200 moves to a step 210 if the processor 110 determines the response as a positive response. At the step 210, the processor 110 disqualifies one or more additive ingredients out of the plurality of additive ingredients for consumption by the patient. For example, naltrexone is disqualified if the patient is on narcotic or opioid pain medications, or has severe allergies to opioid drugs.

Thereafter, the method moves to a step 212, in which the processor 110 selects another question remaining in the plurality of questions for display to the patient. Before selecting the question, the processor 110 may optionally skip/eliminate one or more questions out of the plurality of questions that are directed to disqualify any previously disqualified additive ingredients. For example, the processor 110 may skip a question corresponding to determining if the patient is taking any immunosuppressant drugs if naltrexone was already disqualified on the basis that the patient already answered that the patient has severe allergies to opioid drugs. Therefore, the processor 110 selects the questions that are intended to disqualify one or more of remaining additive ingredients of the variable composition. Thereafter, the method 200 moves to back to the step 204 at which the processor 110 displays the next selected question to the patient.

Also, the method 200 moves to a step 214 if the processor 110 analyzes that the response of the patient is negative at step 208. For example, the method 200 moves to the step 214 if the processor 110 determines that the patient is not on narcotic or opioid pain medications, and does not have severe allergies to opioid drugs. At step 214, the processor 110 selects another question from the remaining questions in the plurality of questions. Thereafter, the method moves back to step 204, in which the processor 110 displays the question selected at step 214.

Once the processor 110 has received responses to all the remaining questions (or alternatively eliminated any remaining questions based on prior responses from the patient), one or more of the additive ingredients that have not been disqualified will be added to the medication, and the resulting customized medication is prescribed to the patient. Accordingly, the method 110 facilitates in identifying the customized weight loss medication based on health history, including but not limited to, pre-existing conditions, existing prescriptions to other medications, drug allergies, and health history.

Further, the method 200 may optionally include determining responses to a second plurality of questions to collect data on the subjective challenges the patient has experienced during their previous weight loss attempts. The data facilitates in determining which of the safe compositions would address the majority of the patients. Patients may then contact a physician to review the recommended formula and to write an initial two-month prescription. The patient is also registered for a screening lab panel and will receive a lab requisition for performing one or more lab tests. The lab tests may include CBC, CMP, and lipid panel, which will provide data to the physician on common co-morbidities associated with obesity, cholesterol levels, and blood sugar levels. Following the initial 2-month period, patients are rescheduled for both a prescription renewal and interpretation of the lab values. Based on the lab interpretation additional prescription intervention may be indicated, and the desired prescription will be compounded into the initial formula.

It should be understood that the foregoing description is only illustrative of the aspects of the disclosed embodiments. Various alternatives and modifications can be devised by those skilled in the art without departing from the aspects of the disclosed embodiments. Accordingly, the aspects of the disclosed embodiments are intended to embrace all such alternatives, modifications, and variances that fall within the scope of the appended claims. Further, the mere fact that different features are recited in mutually different dependent or independent claims does not indicate that a combination of these features cannot be advantageously used, such as a combination remaining within the scope of the aspects of the disclosed embodiments.

What is claimed is:

1. A method for determining a customized medication for weight-loss, the customized medication includes a variable composition, wherein the variable composition includes one or more additive ingredients selected from a plurality of additive ingredients, the method comprising:
displaying, by a display, a plurality of questions to a user;
receiving, by an input device, user responses of the user corresponding to each question in the plurality of questions;
analyzing, by a processor, the user responses; and
disqualifying, by the processor, any of the additive ingredients in the plurality of additive ingredients based on the user responses.

2. The method of claim 1 wherein the plurality of additive ingredients include naltrexone, yohimbine, bupropion, topiramate, phentermine, and metformin.

3. The method of claim 2 wherein each of the questions in the plurality of questions relates to whether the user should be disqualified from using one or more of the additive ingredients of the variable composition.

4. The method of claim 3 including the step of creating, by the processor, of a customized medication for the user once a user response has been received for each of the questions in the plurality of questions, the customized medication including any of the additive ingredients in the variable composition which have not been disqualified by the processor.

5. The method of claim 1 wherein each of the questions in the plurality of questions relates to whether the user should be disqualified from using one or more of the additive ingredients of the variable composition.

6. The method of claim 5 including the step of creating, by the processor, of a customized medication for the user once a user response has been received for each of the questions in the plurality of questions, the customized medication including any of the additive ingredients in the variable composition which have not been disqualified by the processor.

7. The method of claim 1 including the step of creating, by the processor, of a customized medication for the user once a user response has been received for each of the questions in the plurality of questions, the customized medication including any of the additive ingredients in the variable composition which have not been disqualified by the processor.
